(19) United States
(12) Patent Application Publication
(10) Pub. No.: US 2009/0131868 A1
(43) Pub. Date: May 21, 2009

(54) Balloon catheters and related methods

(76) Inventor: Gladwin S. Das, St. Paul, MN (US)

Correspondence Address: CYR & ASSOCIATES, P.A., 605 U.S. Highway 169, Suite 300, Plymouth, MN, 55441, US

(21) Appl. No.: 11/726,115

(22) Filed: Mar. 20, 2007

(51) Int. Cl. A61M 25/10

(52) U.S. Cl. 604/101.01

(57) ABSTRACT

Balloon catheters and stent delivery systems for medical treatment of a patient are disclosed. The balloon catheter includes a hub, a shaft and a first balloon and a second balloon. The first distal balloon and the second balloon may have different lengths. The first distal balloon and the second balloon may have different diameters. The first balloon and the second balloon may be configured to receive a bifurcated stent.

### BACKGROUND OF THE INVENTION

1. Field of the Invention

The present invention relates to cardiovascular therapy and, more particularly, to apparatus and methods for the treatment of vessel of bifurcations.

2. Description of the Related Art

Balloon catheters are used in a variety of therapeutic applications, including intravascular catheters for procedures such as angioplasty. By way of example, the present invention will be described in relation to coronary and peripheral angioplasty treatments. However, the present invention relates to any balloon catheter and stent delivery system having enhanced stent retention, and is not limited to angioplasty.

Most balloon catheters have an elongated flexible shaft defining one or more passages or lumens with one or more inflatable balloons attached near or at one end of the shaft. For reference, the end of the catheter including the balloon will be referred to as the “distal” end, while the other end is called the “proximal” end. The relative position of various components may also be referred to as “proximal” and “distal” based on their position generally along the longitudinal axis of the shaft or more generally based on the distance from the proximal end when the shaft is straightened out to a substantially linear configuration. The balloons may be connected to one or more inflation lumen extending through the shaft for the purpose of selectively inflating and deflating the balloon. The other end of the inflation lumen and other lumen within the shaft may be in fluid communication with a hub to couple the lumen to various devices.

One method for using a balloon catheter is to advance its distal end into the body of a patient, by directing the distal end of the balloon catheter percutaneously through an incision and into a body passage such as a blood vessel. The distal end of the balloon catheter is advanced until the balloon is positioned at a target location. After the balloon is disposed within the target location, the balloon may be inflated to press outward on the body passage. The pressure may be relatively high pressure when the material from which the balloon is formed is an inelastic or non-compliant.

This outward pressing of a constriction or narrowing at the desired site in a body passage is intended to partially or completely re-open or dilate that body passageway or lumen, increasing its inner diameter or cross-sectional area. In the case of a blood vessel, this procedure is referred to as angioplasty. The objective of an angioplasty is to increase the inner diameter or cross-sectional area of the vessel passage or lumen to allow blood to flow more easily through the effected region. The narrowing of the body passageway lumen is typically called a lesion or stenosis, and may be in the form of hard plaque or viscous thrombus.

Unfortunately, the lumen at the angioplasty site may re-close or become narrow again. This will typically occur at around six months after the angioplasty procedure. This narrowing phenomenon is called restenosis. Restenosis occurs in as many as 30-40% of percutaneous transluminal angioplasty patients. Restenosis may require additional procedures, such as another angioplasty, drug therapy treatment, or even surgery including bypass graft. It is generally desirable to prevent or limit the occurrence of restenosis. This is particularly the case in patients whose poor health or other conditions may not make them the preferred candidates for repeated interventional treatment.

In an effort to prevent restenosis, short flexible cylinders or scaffolds made of metal or polymers, referred to as a stent, may be permanently implanted into the vessel to hold the lumen open, to reinforce the vessel wall and improve blood flow. Stents tend to keep the blood vessel open longer. The efficacy of stents has been improved in recent years by the addition of drug coatings which inhibit restenosis as well as other problems associated with the implantation of stents. Unfortunately, the use of stents can be limited by various factors, including size and location of the blood vessel, a complicated or tortuous vessel pathway, bifurcations in the blood vessels etc.

Some stents are expanded to the proper size by inflating a balloon catheter, referred to as “balloon-expandable” stents, while others are designed to elastically resist compression in a “self-expanding” manner. Both balloon-expandable stents and self-expanding stents are generally crimped or compressed to a diameter during delivery that is smaller than the eventual deployed diameter at the desired site. When positioned at the desired site within the lesion, they are deployed by inflating a balloon or being allowed to self-expand into the desired diameter.

### SUMMARY OF THE INVENTION

The present inventions satisfy needs and provide improvements and advantages in the area of balloon catheters that will be recognized by those skilled in the art upon review of the present disclosure.

The present inventions may include a balloon catheter having a hub, a shaft, a first balloon and a second balloon. The shaft may have a proximal end and a distal end. The proximal end may be secured to the hub and the distal end may be secured to the first balloon and the second balloon. The first balloon and the second balloon may have different diameters. The first distal arm may define a first distal diameter. In one aspect, the diameter of the first balloon and the second balloon are substantially the same. In another aspect, the diameter of the first balloon at least 1.2 times larger than the diameter of the second balloon. In one aspect, the first balloon is longer than the second balloon. In one aspect, the shaft defines one or more inflation lumen in which a first inflation tube and a second inflation tube are positioned. The first inflation tube and the second inflation tube may be in fluid communication with a first inflation chamber and a second inflation chamber of the first balloon and the second balloon, respectively. In another aspect, the shaft defining an inflation lumen in fluid communication with a first inflation chamber and a second inflation chamber of the first balloon and the second balloon, respectively. A stent may be secured over at least a portion of one or more of the first balloon and the second balloon.

### BRIEF DESCRIPTION OF THE FIGURES

FIG. 1 illustrates a perspective view of an exemplary embodiment of a balloon catheter in accordance with aspects of the present inventions;

FIG. 2 illustrates a cross section of an exemplary embodiment of a balloon catheter in accordance with aspects of the present inventions in a deflated configuration with a stent secured about the first balloon and the second balloon;

FIG. 3 illustrates a cross section of an exemplary embodiment of a balloon catheter in accordance with aspects of the present inventions having the first balloon and the second balloon in at least a partially inflated configuration;

FIG. 4 illustrates a side view of an exemplary embodiment of a balloon catheter in accordance with aspects of the present inventions in at least a partially inflated configuration;

FIG. 5 illustrates a side view of another exemplary embodiment of a balloon catheter in accordance with aspects of the present inventions in at least a partially inflated configuration;

FIG. 6A illustrates a cross-section through an exemplary embodiment of the balloon catheter in accordance with aspects of the present inventions through section lines 6A-6A of FIG. 1;

FIG. 6B illustrates a cross-section through an exemplary embodiment of the balloon catheter in accordance with aspects of the present inventions through section lines 6B-6B of FIG. 1;

FIG. 6C illustrates a cross-section through an exemplary embodiment of the balloon catheter in accordance with aspects of the present inventions through section lines 6C-6C of FIG. 1; and

FIG. 6D illustrates a cross-section through an exemplary embodiment of the balloon catheter in accordance with aspects of the present inventions through section lines 6D-6D of FIG. 1.

All Figures are illustrated for ease of explanation of the basic teachings of the present invention only; the extensions of the Figures with respect to number, position, relationship and dimensions of the parts to form the embodiment will be explained or will be within the skill of the art after the following description has been read and understood. Further, the exact dimensions and dimensional proportions to conform to specific force, weight, strength, flow and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when the terms “proximal,” “distal,” and similar positional terms are used, the terms should be understood to reference the structures shown in the drawings as they will typically be utilized by a physician or other user who is treating or examining a patient with an apparatus in accordance with the present invention.

### DETAILED DESCRIPTION OF THE INVENTION

Aspects of a balloon catheter system, a balloon catheter and balloon configuration in accordance with aspects of the present inventions are illustrated in exemplary embodiments throughout the attached figures. A balloon catheter in accordance with the present invention has been generally designated “10”.

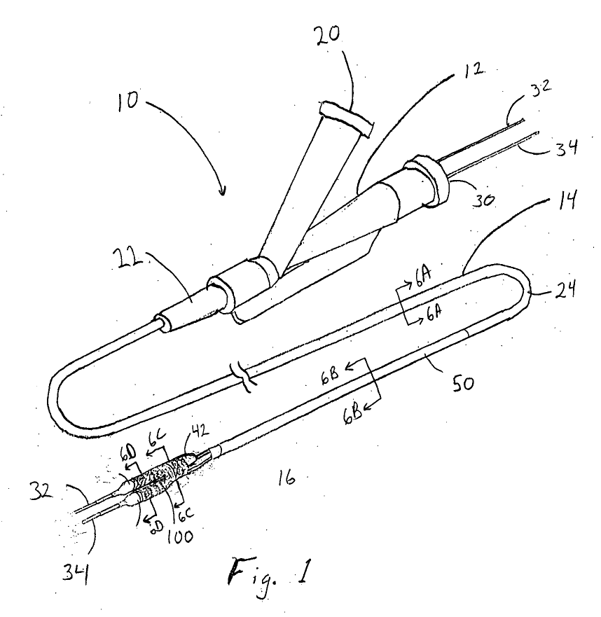

As illustrated in FIG. 1, a balloon catheter 10 in accordance with the present invention may include a hub 12, an elongated and flexible tubular shaft 14, and a first inflatable balloon 116 and a second inflatable balloon 216. The first balloon 116 and the second balloon 216 are typically secured to the shaft 14 near a distal end of the shaft 14, and the hub 12 is affixed toward or at the proximal end of the shaft 14. The first balloon 116 and the second balloon 216 are typically inflatable. The hub 12 and the shaft 14 are typically configured to independently or simultaneously inflate the first balloon 116 and the second balloon 216.

The shaft 14 is configured to permit the controlled positioning of the first balloon 116 and second balloon 216 as well as a conduit for communication with the first balloon 116 and the second balloon 216 while the first balloon 116 and second balloon 216 are advance through the vasculature or other lumen of a patient. The shaft 14 may be configured to be steerable or otherwise directable by a user as will be recognized by those skilled in the art. In one aspect, the shaft 14 may include a braided tube 24 over at least a portion of the length of the shaft 14. The steerability or directability of the shaft 14 provided by the braided tube 24 or otherwise may simplify the navigation through a passage or lumen within a patient. The shaft may include electronics or fiber optics (not shown) to transmit images or other information from within the patient to a user. The shaft 14 can define one or more passages or lumens extending through the shaft 14, one of which may be an inflation lumen 18. In other aspects, the shaft 14 may enclose at least a portion of one or more inflation tubes 114, 214. A first inflation tube 114 typically defines a first inflation lumen 118 and a second inflation tube 214 typically defines a second inflation lumen 218. One or more of the inflation lumen 118, 218 of the inflation tubes 118, 218 may be in fluid communication with the inflation lumen 18 or may be in direct communication with one or more inflation ports 20 on the hub 12. In one aspect, a distal end of the inflation lumen 18 in fluid communication with a first inflation chamber 124 of the first balloon 116 and a second inflation chamber 224 of the second balloon 216. In another aspect, distal ends of at least one of the inflation lumen 118, 218 of the inflation tubes 114, 214 are in fluid communication with one or more of a first inflation chamber 124 of the first balloon 116 and a second inflation chamber 224 of the second balloon 216. The inflation lumen 18 and/or the inflation lumen 118, 218 may be in fluid communication with the first inflation chamber 124 and the second inflation chamber 224 at the distals end of the inflation lumen 18 and/or the inflation lumen 118, 218. The proximal end of the inflation lumen 18 and/or the inflation lumen 118, 218 may be in fluid communication with a pump or other source of pressurized fluid for the purpose of inflating the first balloon 116 and second balloon 216. In one aspect, the pressurized fluid may be dry nitrogen.

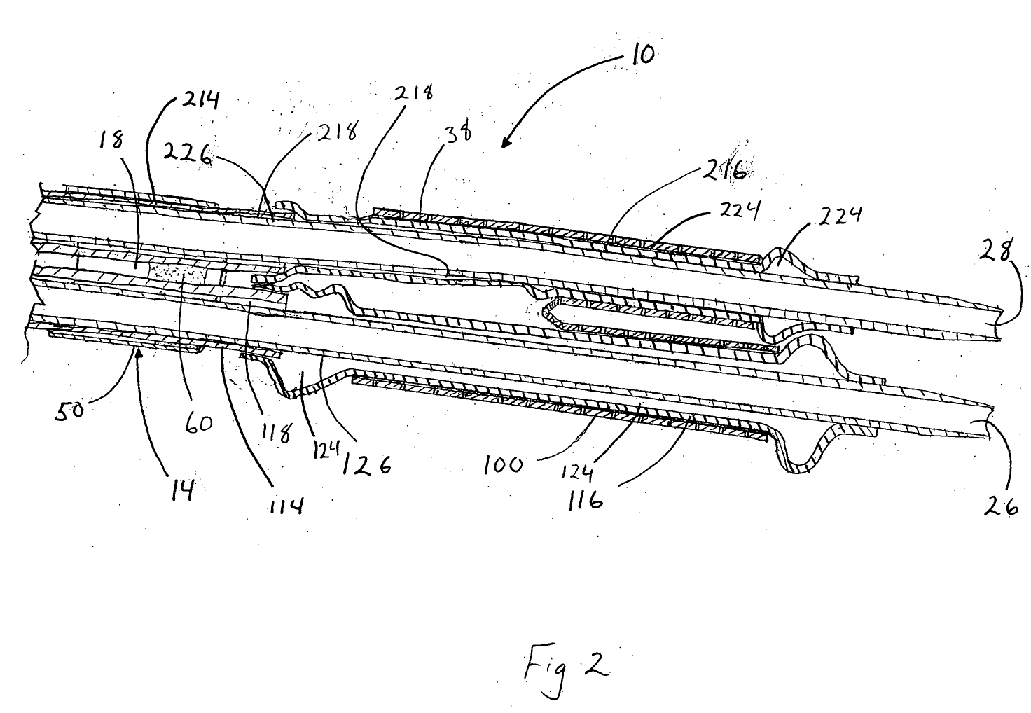

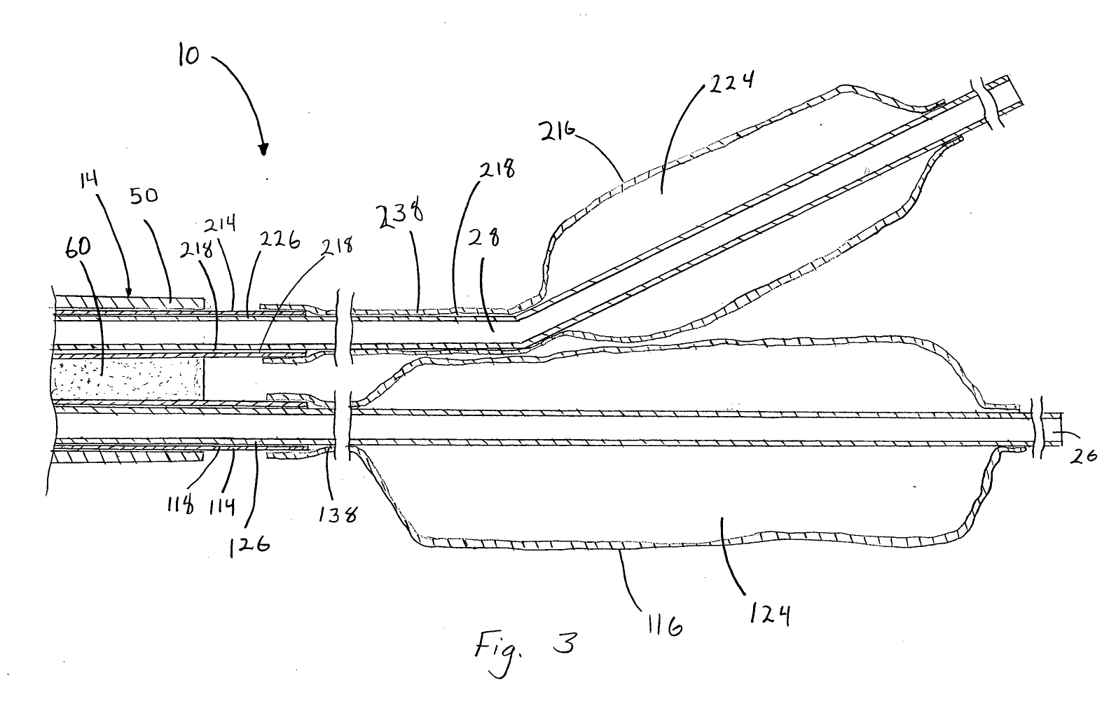

The hub 12 is affixed toward or at the proximal end of the shaft 14. The hub 12 may generally form the point of articulation for a user of the balloon catheter 10. The shaft 14 is generally secured to or within the hub 12. The hub 12 and adjacent portion of shaft 14 may include a strain relief 22. The hub 12 of a balloon catheter 10 typically includes one or more inflation ports 20 having couplings, such as a Luer-lock type fittings, for connecting the inflation lumen 18 and/or the inflation lumen 118, 218 to a source of pressurized fluid. The hub 12 also typically includes one or more guidewire ports 30. The guidewire ports 30 may be in communication with guidewire lumen 26, 28, defined by the shaft 14 or by guidewire tubes 126, 128 extending through shaft 14, shown in FIGS. 2 and 3. The guidewire lumen 26, 28 are generally configured to receive one or more guidewires 32, 34 over which the balloon catheter 10 is passed during a procedure to position the first balloon 116 and second balloon 216 within a lumen of a patient. The one or more guidewire ports 30 typically include one or more hemostatic valves when used in cardiovascular applications. Such valves can allow the guidewires 32, 34 to be extended through the guidewire lumen 26, 28, while resisting the loss of blood or other fluids through the guidewire lumen 26, 28 and guidewire ports 30.

The first balloon 116 and second balloon 216 is generally configured to expand from an uninflated configuration to an inflated configuration. Depending on the application, the balloons 116, 216 may be configured as compliant and/or as non-compliant. The balloons 116, 216 may be constructed of a variety of different materials, including for example Nylon, PEEK, Pebax, silicone among other materials or combinations of materials.

The first balloon 116 generally defines a first length 120 and a first diameter 122 between a first end 124 and a second end 126 of the first balloon 116. The second balloon 216 generally defines a second length 220 and a second diameter 222 between a first end 224 and a second end 226 of the second balloon 216. In certain aspects, second length 220 of the second balloon 216 may be shorter than the first length 120 of the first balloon 116. In other aspects, the diameters 122, 222 of the first balloon 116 and the second balloon 216 may be different. For example, the first diameter 122 of the first balloon 116 may be less than the second diameter 222 of the second balloon 216. In certain aspects, the cross sectional shapes and/or diameters 122, 222 of the balloons 116, 216 may vary along their lengths 120, 220.

The first balloon 116 and second balloon 216 may include a bifurcated stent 100 or at least two separate stents 100 secured about at least portions of the first balloon 116 and second balloon 216. The stents 100 may be secured over the first balloon 116 and second balloon 216 while over the first balloon 116 and second balloon 216 are in an un-inflated configuration. The stents 100 may be particularly configured to be deployed at a vessel bifurcation. A bifurcated stent 100 may generally secured such that the one of the branches of the bifurcated stent 100 is positioned over the first balloon 116 and the other branch of the bifurcated stent 100 is positioned over the second balloon 216. When the first balloon 116 and second balloon 216 are inflated, the bifurcated stent 100 is typically expanded and will typically remain expanded after the first balloon 116 and second balloon 216 are deflated. Similarly, two separate stents 100 may generally positioned over the first balloon 116 and the second balloon 216 such that a first stent 100 is positioned over the first balloon 116 and a second stent 100 is positioned over the second balloon 216. When the first balloon 116 and second balloon 216 are inflated, simultaneously or sequentially, each of the first and second stents 100 are typically expanded and will typically remain expanded after the first balloon 116 and second balloon 216 is deflated. Accordingly, the bifurcated stent 100 or the at least two separate stents 100 may be retained the vessel in an open position after the balloon configuration is deflated and removed from the target location.

As illustrated for exemplary purposes in FIGS. 2 and 3, at least the distal end of the shaft 14 may include a tube 50, a first inflation tube 124, a first guidewire tube 126, a second inflation tube 224, and a second guidewire tube 226. The inflation lumen 18 may be defined between the guidewire tubes 126, 226 and tube 50 as illustrated in the exemplary embodiment. In one aspect, the inflation lumen 18 may communicate with each of the first balloon 116 and the second balloon through the first inflation lumen 118 and the second inflation lumen 218, respectively. In another aspect, a first inflation tube 114 extends between at least the inflation lumen 18 and a first inflation chamber of the first balloon 116.

First guidewire tube 126 and second guidewire tube 226 may extend through the entire length or along a portion of the length of the shaft 14. First guidewire tube 126 and second guidewire tube 226 may extend through the entire lengths 120, 220 or along a portion of the lengths 120, 220 of the first balloon 116 and second balloon 216, respectively. As illustrated, the first guidewire tube 126 and the second guidewire tube 226 extend through the first balloon 116 and second balloon 216, respectively. The first inflation tube 124 is positioned over at least a portion of the first guidewire tube 126 with the first inflation lumen 118 of the first inflation tube 124 in communication with the first inflation chamber 124 of the first balloon 116. The second inflation tube 224 is positioned over at least a portion of the second guidewire tube 226 with the second inflation lumen 218 of the second inflation tube 224 in communication with the second inflation chamber 224 of the second balloon 216. The distal portions of first balloon 116 and second balloon 216 are typically sealingly secured about the first guidewire tube 126 and the second guidewire tube 226 to permit the first balloon 116 and second balloon 216 to inflate upon receiving a fluid through one or more of inflation lumen 18 and/or inflation lumen 118, 218. The first guidewire tube 126 and the second guidewire tube 226 define a first guidewire lumen 26 and a second guidewire lumen 28, respectively. The guidewire lumen 26, 28 are generally adapted to receive one or more elongated flexible guidewires 32, 34 in a sliding fashion, such that the guidewire 32, 34 and balloon catheter 10 may be advanced or withdrawn independently, and/or the balloon catheter 10 may be guided along a path selected with the guidewire 32, 34. The shaft 14 may have various configurations distinct from those illustrated in the figures, including a single extruded tube defining any suitable number of parallel or spiraling side-by-side lumens, among other configurations.

The inflation lumen 18 and/or inflation lumen 118, 218 may be configured to communicate fluid between a source of pressurized fluid and at least one of interior 22 of the first balloon 116 and second balloon 216 and. Accordingly, first balloon 116 and second balloon 216 is typically sealing secured about inflation lumen 18. The balloon configuration is typically secured to shaft 14. As illustrated, the first balloon 116 and second balloon 216 are secured to the tube 50 with the first balloon 116 and second balloon 216 to be in fluid communication with the inflation lumen 18 of shaft 14 through. The pressurized fluid is received from the pump or other source, not shown, and conveyed through the inflation lumen 18 to the interior chamber 22 of the first balloon 116 and second balloon 216.

FIG. 2 illustrates a first balloon 116 and second balloon 216 in an un-inflated configuration. As illustrated, a bifurcated stent 100 is secured about the first balloon 116 and second balloon 216. The assembly of the bifurcated stent 100 and the first balloon 116 and second balloon 216 is in a substantially linear configuration to permit its introduction through a lumen or passage in the patient. Once positioned over on or more guidewires 32, 34 within a patient, a pressurized fluid is introduced into the first inflation chamber 124 of the first balloon 116 and the second inflation chamber 124 of the second balloon 216. Typically, the fluid is introduced either sequentially or simultaneously but fluid introduction may be varied as will be recognized by those skilled in the art upon review of the present disclosure. This introduction or forcing of fluid into the first balloon 116 and second balloon 216 tends to inflate the balloons 116, 216. Inflation may occur by expanding the material of the first balloon 116 and second balloon 216 as is typical of certain compliant balloons, unfolding pleats in the material of the first balloon 116 and second balloon 216 as is typical of certain non-compliant balloons, or by a combination thereof. As particularly illustrated, the tube 50 includes a first guidewire tube 126 and a second guidewire tube 226 extending from a distal end of tube 50. The first guidewire lumen 26 extends to the distal end of the first guidewire tube 126. The second guidewire lumen 28 extends to the distal end of the second guidewire tube 226.

As particularly illustrated in FIG. 2, the first guidewire tube 126 is positioned coaxially within the lumen of the first inflation tube 114. The space between the outer surface of the first guidewire tube 126 and the inner surface of the first inflation tube 114 define the first inflation lumen 118. As illustrated, the first inflation lumen 118 communicates fluid directly into the first inflation chamber 124 of the first balloon 116.

As particularly illustrated in FIG. 2, the second guidewire tube 226 is positioned coaxially within the lumen of the second inflation tube 214. The space between the outer surface of the second guidewire tube 226 and the inner surface of the second inflation tube 214 define the second inflation lumen 218. A distal portion of the second inflation lumen 218 is defined by a space between the outer surface of the second guidewire tube 226 and the inner surface of a second distal tube 238 which is integrally formed with the material of the second balloon 216 and, for exemplary purposes, enlarges to form the second inflation chamber 224 at the proximal end of the second balloon. Accordingly, a distal end of the second inflation tube 214 is secured in fluid communication with the proximal end of the second balloon 216. As illustrated, the second inflation lumen 218 communicates fluid through a distal portion of the second inflation lumen 218 defined by the material of the second balloon 216 into the second inflation chamber 224 of the second balloon 216.

As particularly illustrated in FIG. 2, a distal end of the inflation lumen 18 defined by tube 50 is sealed with a sealant 60. The inflation lumen 18 may be in fluid communication with the first inflation lumen 118 of the first inflation tube 114 and the second inflation lumen 218 of the second inflation tube 214 at a location proximal to the illustration that is not shown.

FIG. 3 illustrates a first balloon 116 and second balloon 216 similar to the embodiment of FIG. 2 in an at least partially inflated configuration without an associated stent 100. The first balloon 116 and the second balloon 216 are shown having had fluid introduced or forced into the first inflation chamber 124 and second inflation chamber 224 of the respective balloons. The fluid has at least partially inflated both the first balloon 116 and the second balloon 216. In the illustrated inflated configuration, the first balloon 116 and second balloon 216 are oriented in a Y-shaped configuration similar to an orientation in bifurcation in a lumen or passage within a patient. As particularly illustrated, the tube 50 includes a first guidewire tube 126 and a second guidewire tube 226 extending from a distal end of tube 50. The first guidewire lumen 26 extends to the distal end of the first guidewire tube 126. The second guidewire lumen 28 extends to the distal end of the second guidewire tube 226.

As particularly illustrated in FIG. 3, the first guidewire tube 126 is positioned coaxially within the lumen of the first inflation tube 114. The space between the outer surface of the first guidewire tube 126 and the inner surface of the first inflation tube 114 define the first inflation lumen 118. A distal portion of the first inflation lumen 118 is defined by a space between the outer surface of the first guidewire tube 126 and the inner surface of a distal tube 138 which is integrally formed with the material of the second balloon 116 and, for exemplary purposes, enlarges to form the first inflation chamber 124 at the proximal end of the first balloon 116. Accordingly, a distal end of the first inflation tube 114 is secured in fluid communication with the proximal end of the first balloon 116. As illustrated, the second inflation lumen 218 communicates fluid through a distal portion of the first inflation lumen 118 defined by the material of the first balloon 116 into the first inflation chamber 124 of the first balloon 116.

As particularly illustrated in FIG. 3, the second guidewire tube 226 is positioned coaxially within the lumen of the second inflation tube 214. The space between the outer surface of the second guidewire tube 226 and the inner surface of the second inflation tube 214 define the second inflation lumen 218. A distal portion of the second inflation lumen 218 is defined by a space between the outer surface of the second guidewire tube 226 and the inner surface of a second distal tube 238 which is integrally formed with the material of the second balloon 116 and, for exemplary purposes, enlarges to form the second inflation chamber 224 at the proximal end of the second balloon. Accordingly, a distal end of the second inflation tube 214 is secured in fluid communication with the proximal end of the second balloon 216. As illustrated, the second inflation lumen 218 communicates fluid through a distal portion of the second inflation lumen 218 defined by the material of the second balloon 216 into the second inflation chamber 224 of the second balloon 216.

As particularly illustrated in FIG. 3, a distal end of the inflation lumen 18 defined by tube 50 is sealed with a sealant 60. The inflation lumen 18 may be in fluid communication with the first inflation lumen 118 of the first inflation tube 114 and the second inflation lumen 218 of the second inflation tube 214 at a location proximal to the illustration that is not shown.

FIGS. 4 and 5 illustrate two exemplary embodiments of a first balloon 116 and second balloon 216 that include aspects of the present inventions. In one aspect, the first guidewire tube 126 and the second guidewire tube 128 are illustrated with at least one marker band 170 extending about them. The marker bands 170 illustrated in phantom are positioned about the first guidewire tube 126 and second guidewire tube 128 within the first inflation chamber 124 of the first balloon 116 and the second inflation chamber 224 of the second balloon 216, respectively. The balloon configurations of FIGS. 4 and 5 include a first balloon 116 and a second balloon 216 secured to the distal end of a shaft 50. The first balloon 116 include has a generally circular profile between a first proximal end 146 and a first distal end 156 of the first balloon 116. The second balloon 216 includes a second proximal end 246 and a second distal end 256. As illustrated in FIG. 5, the second balloon 216 may include a flattened region 266 at the second proximal end 246. The flattened region 266 may be configured to conform to an outer surface of the first balloon 116 when both the first balloon 116 and the second balloon 216 are inflated. Each of the illustrated embodiments includes a second balloon 216 that is shorter than the first balloon 116. Particularly, the second proximal end 246 of the second balloon 216 is configured to be positioned distal to the first proximal end 146 of the first balloon 116. In certain embodiments, the second distal end 256 of the second balloon 216 and the first distal end 156 of the first balloon 116 are generally configured to extend to substantially the same point along the longitudinal axis of the balloon catheter 10 at least when the first balloon 116 and the second balloon 216 are uninflated. As illustrated in FIG. 4, the first inflation tube 114 and the second inflation tube 214 may independently extend from the distal end of shaft 50 to the first proximal end 146 of the first balloon 116 and the second proximal end 246 of the second balloon 216, respectively. As illustrated in FIG. 5, the first inflation tube 114 and the second inflation tube 214 may be secured to one another or unitarily constructed between at least a portion of the distal end of shaft 50 and the first proximal end 146 of the first balloon 116. As further illustrated in FIG. 5, the second inflation tube 214 may be secured to an outer surface of the first balloon 116 or may be integral with the first balloon 116 between at least a portion of the first proximal end 146 of the first balloon 116 and the second proximal end 246 of the second balloon 216.

As illustrated in FIGS. 1 to 5, the first length 120 of the first balloon 116 may be longer than the second length 220 of the second balloon 216. In one aspect, the first length 120 may be approximately 1.1 times the length of the second length 220. In another aspect, the second length may be 1.2 times the length of the second length 220. In another aspect, the second length may be 1.3 times the length of the second length 220. In another aspect, the second length may be 1.4 times the length of the second length 220. In another aspect, the second length may be 1.5 times the length of the second length 220. In another aspect, the second length may be at least 1.5 times the length of the second length 220.

As is further illustrated in FIGS. 1 to 5, the first diameter 122 of the first balloon 116 may be greater than the second diameter 222 of the second balloon 216. In another aspect of the present invention, the first diameter 122 may be equal to or substantially equal to the second distal diameter 80. In another aspect, the first diameter 122 may be at least 1.2 times larger than the second diameter 222.

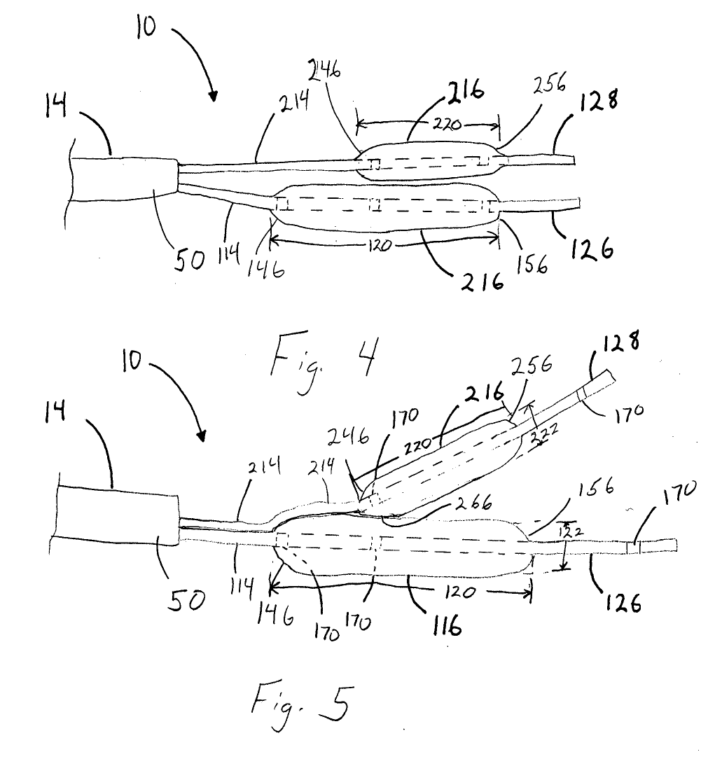

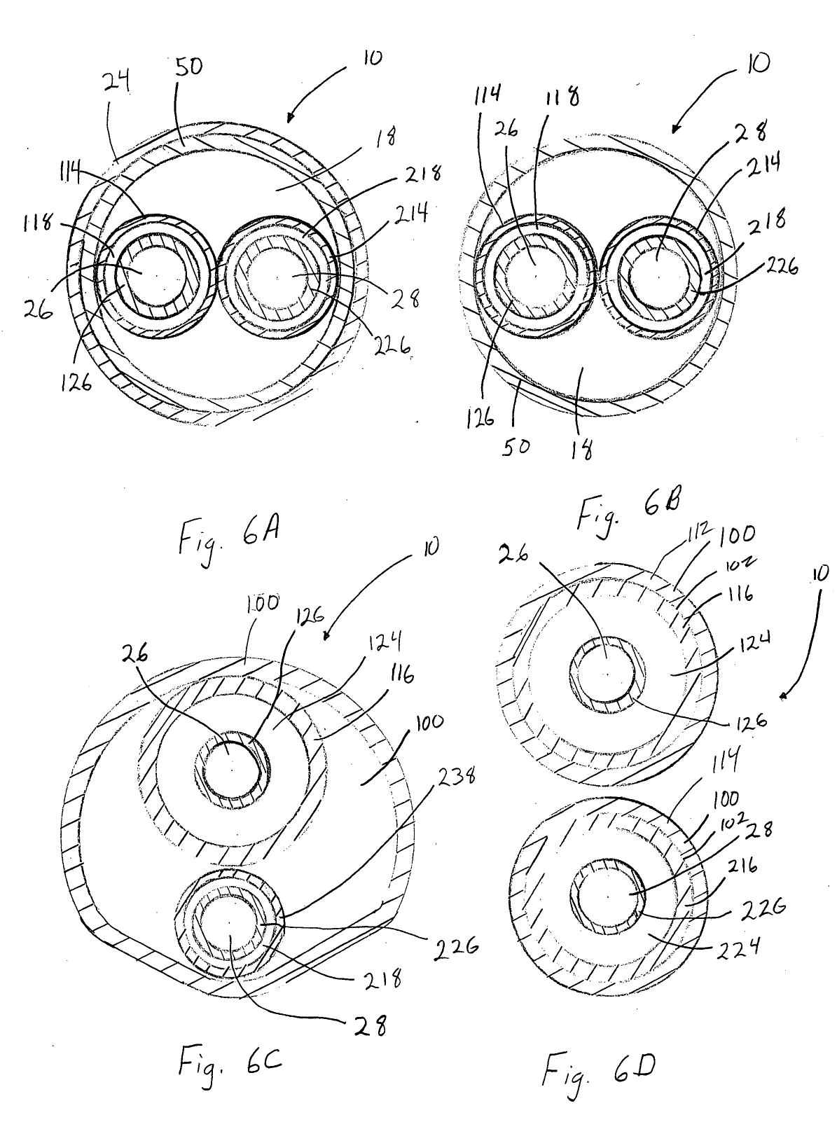

As illustrated in FIG. 6A for exemplary purposes, a tube 50 may include a braided tube 24 extending about the outer surface of the tube 50 at section line 6A-6A of FIG. 1. The braided tube 24 may be secured to or slidably received over the outer surface of tube 50. The tube 50 may further define an inflation lumen 18 through at least the intermediate portion of balloon catheter 10. In certain aspect, the inflation lumen 18 may be in fluid communication with an inflation port 20 of a hub 12. In other aspects, the inflation lumen 18 is not in fluid communication with the inflation port 20 of hub 12. In other aspects, the inflation lumen 18 is configured to receive or define one or more of the first inflation tube 114 and the second inflation tube 214. The first inflation tube 114 and the second inflation tube 218 may be positioned and/or secure in the inflation lumen 18 in a substantially parallel configuration. The first inflation tube 114 defines a first inflation lumen 118. The first inflation lumen 118 is generally configured to communicate a fluid from the proximal end of the first inflation lumen 118 to the distal end of the first inflation lumen 118. The first inflation lumen 118 is in either direct or indirect communication with at least one inflation port 20 to permit the introduction of a fluid for inflation of the first balloon 116. In certain aspects, the first guidewire tube 126 may be received through the first inflation lumen 118. As further illustrated, the first guidewire lumen 26 extends longitudinally through the first inflation lumen 118. Similarly, the second inflation lumen 218 is generally configured to communicate a fluid from the proximal end of the second inflation lumen 218 to the distal end of the second inflation lumen 218. The second inflation lumen 218 is in either direct or indirect communication with at least one inflation port 20 to permit the introduction of a fluid for inflation of the second balloon 216. In certain aspects, the second guidewire tube 226 may be received through the second inflation lumen 218. As further illustrated, the second guidewire lumen 28 extends longitudinally through the second inflation lumen 218.

As illustrated in FIG. 6B for exemplary purposes, a tube 50 may define an inflation lumen 18 through at least a distal portion of the balloon catheter 10 at section line 6B-6B of FIG. 1. In certain aspect, the inflation lumen 18 may be in fluid communication with an inflation port 20 of a hub 12. In other aspects, the inflation lumen 18 is not in fluid communication with the inflation port 20 of hub 12. In other aspects, the inflation lumen 18 is configured to receive or define one or more of the first inflation tube 114 and the second inflation tube 214. The first inflation tube 114 and the second inflation tube 218 may be positioned and/or secure in the inflation lumen 18 in a substantially parallel configuration. The first inflation tube 114 defines a first inflation lumen 118. The first inflation lumen 118 is generally configured to communicate a fluid from the proximal end of the first inflation lumen 118 to the distal end of the first inflation lumen 118. In certain aspects, the first guidewire tube 126 may be received through the first inflation lumen 118. As further illustrated, the first guidewire lumen 26 extends longitudinally through the first inflation lumen 118. Similarly, the second inflation lumen 218 is generally configured to communicate a fluid from the proximal end of the second inflation lumen 218 to the distal end of the second inflation lumen 218. In certain aspects, the second guidewire tube 226 may be received through the second inflation lumen 218. As further illustrated, the second guidewire lumen 28 extends longitudinally through the second inflation lumen 218.

As illustrated in FIG. 6C for exemplary purposes, the first balloon 116 and second distal tube 238 are shown extending through a stent 100 at section line 6C-6C of FIG. 1. The first balloon 116 and the second distal tube 238 extend longitudinally within a passage 102 defined by the stent 100. The first balloon 116 and the second distal tube 238 are typically removably secured within the passage 102. The first balloon 116 is positioned to permit the first balloon 116 to expand a proximal portion of the stent 100. The first inflation chamber 124 is configured to receive a fluid to expand the first balloon 116. The second distal tube 238 is configured to permit the communication of fluid through a second inflation lumen 218 defined between the second distal tube 238 and the second guidewire tube 226 and into the distally positioned second balloon 216.

As illustrated in FIG. 6D for exemplary purposes, a first branch 112 and a second branch 114 of the bifurcated stent 100 are shown with the first balloon 116 and the second balloon 216 secured within the passages 102 at section line 6D-6D of FIG. 1. The first balloon 116 and the second balloon 216 are shown extending longitudinally within passages 102 defined by the first branch 112 and the second branch 114 of stent 100. The first balloon 116 and the second balloon 216 are typically removably secured within the passages 102. The first balloon 116 and the second balloon 216 are shown in an at least partially inflated configuration for exemplary purposes. The first balloon 116 is positioned to permit the first balloon 116 to expand the first branch 112 of the stent 100. The first inflation chamber 124 is configured to receive a fluid to expand the first balloon 116. The second balloon 216 is positioned to permit the second balloon 216 to expand the second branch 114 of the stent 100. The second inflation chamber 224 is configured to receive a fluid to expand the second balloon 216.

As generally illustrated in FIGS. 6A to 6D, the first guidewire lumen 26 defined by the first guidewire tube 126 and the second guidewire lumen 28 defined by the second guidewire tube 226 are each configured to slidably receive a guidewire and to permit the passage of the guidewire through the first guidewire lumen 26 and the second guidewire lumen 28 as will be recognized by those skilled in the art upon review of the present disclosure.

To use a balloon catheter 10 in accordance with the present invention, a user may insert the distal end of a first guidewire 32 and a second guidewire 34 into the desired location within a patient. In certain circumstances, the distal end of the first guidewire 32 is placed within the first branch of a vessels bifurcation and the distal end of the second guidewire 34 is placed within a second branch of the vessels bifurcation. The proximal end of the first guidewire 32 may then be inserted into the first guidewire lumen 26 of the balloon catheter 10. The proximal end of the second guidewire 34 may then be inserted into the second guidewire lumen 28 of the balloon catheter 10. The user may insert the distal end of balloon catheter 10 into a bodily lumen of a patient. As the guidewires 32, 34 slide through the guidewire lumen, the balloon catheter 10 tracks the guidewires 32, 34 and is guided to the desired bifurcation within the patient requiring treatment. As balloon catheter 10 is guided through the patient, a user can manipulate the hub 12 or the proximal end of the tube 50 to direct the distal end of the balloon catheter 10 through the bodily lumen. When the distal end of the balloon catheter 10 is positioned at or near the location within the bodily lumen requiring treatment, the user may remove the guidewires 32, 34 from the patient and balloon catheter 10 and initiate the desired treatment.

In certain applications, balloon catheters 10 may further be used to guide surgical, therapeutic or diagnostic instruments over balloon catheters 10 to access a desired location in a bodily lumen. When the instrument is positioned at the desired location within the bodily lumen, at least one surgical, therapeutic or diagnostic procedure using the instrument is performed. The instrument may be removed and replaced with a different instrument as required by the treatment, diagnosis, or surgical procedure being performed by the user.

The foregoing discussion discloses and describes merely exemplary embodiments of the present invention. Upon review of the specification, one skilled in the art will readily recognize from such discussion, and from the accompanying drawings and claims, that various changes, modifications and variations can be made therein without departing from the spirit and scope of the invention as defined in the following claims.

What is claimed is:

1. A balloon catheter, comprising:
a hub defining an inflation port;
a shaft secured to at a proximal end of the shaft and extending from the hub;
a first inflation tube extending from a distal end of the shaft;
a second inflation tube extending from the distal end of the shaft;
a first balloon having an elongated configuration and a first length; and
a second balloon having an elongated configuration and a second length, the first length is longer than the second length.

2. A balloon catheter, as in claim 1, further comprising the first inflation tube secured to at least a portion of the second inflation tube between a distal end of the shaft and a proximal end of the first balloon.

3. A balloon catheter, as in claim 1, further comprising the second inflation tube secured to at least a portion of the first balloon.

4. A balloon catheter, as in claim 3, further comprising the second inflation tube secured to at least a portion of an outer surface of the first balloon between the first proximal end of the first balloon and the second proximal end of the second balloon.
